(19) United States
(12) Patent Application Publication
(10) Pub. No.: US 2023/0095963 A1
(43) Pub. Date: Mar. 30, 2023

(54) MEDICINE ADMINISTRATION AND TRACKING SYSTEMS AND METHODS INCLUDING CUSTOMIZED USER ALERTS

(71) Applicant: Medtronic MiniMed, Inc., Northridge, CA (US)

(72) Inventors: Madison B. Smith, San Diego, CA (US); Angela R. Gaetano, San Diego, CA (US); Michael R. Mensinger, San Diego, CA (US)

(21) Appl. No.: 17/485,597

(22) Filed: Sep. 27, 2021

(51) Int. Cl. A61J 7/04; G08B 21/24; G08B 25/00; G08B 7/00

(52) U.S. Cl. CPC A61J 7/0418; A61J 7/0481; G08B 21/24; G08B 25/008; G08B 7/00

(57) ABSTRACT

A method, and a system configured to perform a method, for customizing user alerts in a medicine administration and tracking system including detecting an alertable condition for which a medicine administration and tracking system is configured to monitor, generating a custom alert for the alertable condition based at least in part on historical data indicating a responsiveness to previous alerts for the same alertable condition, delivering the generated custom alert to a user, determining the user's response to the generated custom alert, and updating the historical data to include the alertable condition, the generated custom alert, and the user's level of responsiveness to the generated custom alert.

### FIELD

The present disclosure relates to medicine administration and tracking and, more specifically, to medicine administration and tracking systems and methods including customized user alerts based on historical data.

### BACKGROUND

Diabetes mellitus (“diabetes”) is a metabolic disease associated with high blood sugar due to insufficient production or use of insulin by the body. Diabetes affects hundreds of millions of people and is among the leading causes of death globally. Diabetes has been categorized into three types: type 1, type 2, and gestational diabetes. Type 1 diabetes is associated with the body's failure to produce sufficient levels of insulin for cells to uptake glucose. Type 2 diabetes is associated with insulin resistance, in which cells fail to use insulin properly. Gestational diabetes can occur during pregnancy when a pregnant woman develops a high blood glucose level. Gestational diabetes often resolves after pregnancy; however, in some cases, gestational diabetes develops into type 2 diabetes.

Various diseases and medical conditions, such as diabetes, require a user to self-administer doses of medicine. When administering a liquid medicine by injection, for example, the appropriate dose amount is set and then dispensed by the user, e.g., using a syringe, a medicine delivery pen, or a pump. Regardless of the particular device utilized for injecting the liquid medicine, it is important to accurately track the medicine dosed and to facilitate a user's compliance with a dosing regime, particularly for managing lifelong or chronic conditions like diabetes.

### SUMMARY

To the extent consistent, any of the aspects and features detailed herein can be utilized with any of the other aspects and features detailed herein in any suitable combination.

Provided in accordance with aspects of the present disclosure is a method for customizing user alerts in a medicine administration and tracking system. The method includes detecting an alertable condition for which a medicine administration and tracking system is configured to monitor, generating a custom alert for the alertable condition based at least in part on historical data indicating a responsiveness to previous alerts for the same alertable condition, delivering the generated custom alert to a user, determining the user's response to the generated custom alert, and updating the historical data to include the alertable condition, the generated custom alert, and the user's level of responsiveness to the generated custom alert.

In an aspect, generating the custom alert includes determining at least one of a type of device to which the alert is to be delivered or a manner in which the alert is to be delivered.

In an aspect, the generated custom alert is at least one of an audible alert and generating the custom alert includes determining a volume level of the audible alert, or a haptic alert and generating the custom alert includes determining an intensity or vibratory pattern of the haptic alert.

In an aspect, generating the custom alert includes delaying delivery of the alert to the user based on a time of day that the alertable condition is detected.

In an aspect, the method includes delaying delivery of the custom alert based on an activity of the user.

In an aspect, the generated custom alert includes a calculated insulin dose recommendation.

In an aspect, generating the custom alert includes determining for what alert configuration the user is most likely to respond.

In an aspect, the user is clustered into a cluster and generating the custom alert is based on a setting associated with the cluster.

In an aspect, determining the user's response to the generated custom alert includes determining that the user has not responded to the generated custom alert after expiration of a predetermined period of time. Additionally, or alternatively, the method may further include prompting the user for feedback on the failure to respond and updating the historical data of the user based on the feedback.

In another aspect of the disclosure, a non-transitory computer-readable storage medium is provided. The non-transitory computer-readable storage medium stores instructions, which when executed by a processor, cause the processor to detect an alertable condition for which a medicine administration and tracking system is configured to monitor, generate a custom alert for the alertable condition based at least in part on historical data indicating a responsiveness to previous alerts for the same alertable condition, deliver the generated custom alert to a user, determine the user's response to the generated custom alert, and update the historical data to include the alertable condition, the generated custom alert, and the user's level of responsiveness to the generated custom alert.

In an aspect, the instructions, when executed by the processor, cause the processor to generate the custom alert by determining at least one of a type of device to which the alert is to be delivered or a manner in which the alert is to be delivered.

In an aspect, the generated custom alert is at least one of an audible alert and the instructions, when executed by the processor, cause the processor to determine a volume level of the audible alert, or a haptic alert and the instructions, when executed by the processor, cause the processor to determine an intensity or vibratory pattern of the haptic alert.

In an aspect, the instructions, when executed by the processor, cause the processor to generate the custom alert by delaying delivery of the alert to the user based on a time of day that the alertable condition is detected.

In an aspect, the instructions, when executed by the processor, cause the processor to delay delivery of the alert based on an activity of the user.

In an aspect, the generated custom alert includes a calculated insulin dose recommendation.

In an aspect, the instructions, when executed by the processor, cause the processor to generate the custom alert by determining for what alert configuration the user is most likely to respond.

In an aspect, the user is clustered into a cluster and the instructions, when executed by the processor, cause the processor to generate the custom alert based on a setting associated with the cluster.

In an aspect, the instructions, when executed by the processor, cause the processor to determine the user's response to the alert by determining that the user has not responded to the generated custom alert after expiration of a predetermined period of time.

In an aspect, the instructions, when executed by the processor, cause the processor to prompt the user for feedback on the failure to respond and update the historical data of the user based on the feedback.

### BRIEF DESCRIPTION OF DRAWINGS

FIG. 1A is a schematic illustration of a medicine administration and tracking system provided in accordance with the present disclosure including a medicine injection device, a computing device, and, in aspects, a sensor device and/or a data processing system;

FIG. 1B is a block diagram of the medicine injection device of the system of FIG. 1A;

FIG. 1C is a block diagram of the computing device of the system of FIG. 1A;

FIG. 2 is a flowchart illustrating a method for customizing user alerts in accordance with aspects of the disclosure;

FIG. 3 is flowchart illustrating a method for setting a customized alert to be delivered in accordance with aspects of the disclosure;

FIG. 4 is flowchart illustrating a method for setting a customized alert to be delivered in accordance with aspects of the disclosure; and

FIG. 5 is flowchart illustrating a method for setting a customized alert to be delivered in accordance with aspects of the disclosure.

### DETAILED DESCRIPTION

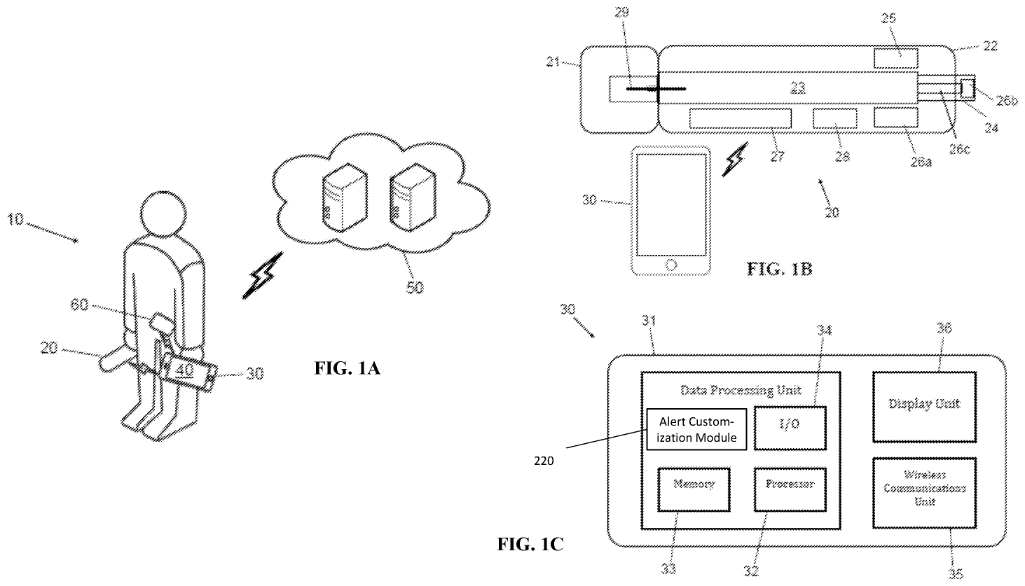

FIG. 1A illustrates a medicine administration and tracking system 10 provided in accordance with the present disclosure including a medicine injection device, e.g., a medicine injection pen 20, in wireless communication with a computing device 30 running a health management application 40 associated with pen 20 and/or other devices part of or connected to system 10. System 10, in aspects, further includes a data processing system 50 and/or a sensor device 60. While the present disclosure is detailed herein with respect to a medicine injection pen 20 and system 10 for diabetes management, it is understood that the present disclosure is also applicable for use with other medicine injection devices, for management of other diseases and medical conditions, and/or for use with other medicine administration and tracking systems.

Medicine injection pen 20, described in greater detail below, is a reusable injection pen configured to removably receive a medicine cartridge, e.g., a cartridge of insulin, for injecting a selected dose of insulin into a patient and recording information concerning the injected dose of insulin, e.g., a dose amount and/or timestamp data associated with the dose.

Computing device 30 is detailed and illustrated herein as a smartphone, although any other suitable computing device may be provided such as, for example, a tablet, a wearable computing device (e.g., a smart watch, smart glasses, etc.), a laptop and/or desktop computer, a smart television, a network-based server computer, etc.

Health management application 40 is paired with pen 20, which may be a prescription-only medical device, via computing device 30, although other suitable configurations are also contemplated. In aspects, the pairing of computing device 30 with pen 20 at least partially unlocks health management application 40 to enable the user to utilize some or all features of health management application 40, e.g., according to the user's prescription. Thus, the act of pairing can unlock and enable the functionality of health management application 40 and/or system 10 (including pen 20), while health management application 40 (and/or system 10) may provide only limited features in the absence of pairing with pen 20.

Health management application 40 of computing device 30, in aspects, can monitor and/or control functionalities of pen 20 and provide a dose calculator module and/or decision support module that can calculate and recommend a dose of medicine for the user to administer using pen 20. Health management application 40 provides a user interface, on the user interface of computing device 30, to allow a user to manage health-related data. For example, health management application 40 can be configured to control some functionalities of pen 20 and/or to provide an interactive user interface to allow a user to manage settings of pen 20 and/or settings for computing device 30 that can affect the functionality of system 10 (FIG. 1A). Computing device 30 can additionally or alternatively be used to obtain, process, and/or display contextual data that can be used to relate to the health condition of the user, including the condition for which pen 20 is used to treat. For example, computing device 30 may be operable to track the location of the user; physical activity of the user including step count, movement distance and/or intensity, estimated calories burned, and/or activity duration; and/or interaction pattern of the user with computing device 30. In aspects, health management application 40 can aggregate and process the contextual data to generate decision support outputs, e.g., on the user interface, to guide and aid the user in monitoring their condition, using pen 20, and/or managing their behavior to promote treatment and better health outcomes. Health management application 40 includes an alert customization module 220, described in greater detail below, which is configured to generate customized alerts for users based at least in part on historical data of the user and/or other similarly grouped users.

In aspects, system 10 further includes a data processing system 50 in communication with pen 20 and/or computing device 30. Data processing system 50 can include one or more computing devices in a computer system and/or communication network accessible via the internet, e.g., including servers and/or databases in the cloud. System 10 can additionally or alternatively include sensor device 60 to monitor one or more health metrics and/or physiological parameters of the user. Examples of health metric and physiological parameter data monitored by sensor device 60 include analytes (e.g., glucose), heart rate, blood pressure, user movement, temperature, etc. Sensor device 60 may be a wearable sensor device such as a continuous glucose monitor (CGM) to obtain transcutaneous or blood glucose measurements that are processed to produce continuous glucose values. For example, the CGM can include a glucose processing module implemented on a stand-alone display device and/or implemented on computing device 30, which processes, stores, and displays the continuous glucose values for the user. Such continuous glucose values can be utilized by health management application 40, for example, for displaying health data, in dose calculation and/or decision support, etc. In aspects, data processing system 50 includes alert customization module 220 in addition to or as an alternative to alert customization module 220 residing on computing device 30.

With reference to FIG. 1B, pen 20 includes a cap 21 configured to protect a medicine dispensing element (e.g., a needle 29) and a body 22 configured to contain a replaceable medicine cartridge 23, e.g., an insulin cartridge. Pen 20 further includes a dose dispensing mechanism 24 to dispense (e.g., deliver) medicine contained in medicine cartridge 23 out of pen 20 (e.g., through needle 29); a dose setting mechanism 25 to enable the selection and/or setting of a dose of medicine to be dispensed; an operations monitoring mechanism 28 (e.g., including one or more switches, sensors (electrical, optical, acoustic, magnetic, etc.), encoders, etc.) to qualitatively determine that pen 20 is being operated and/or to monitor the operation of pen 20 (e.g., to quantitatively determine an amount of medicine set and/or dosed); and an electronics unit 27 that can include a processor, a memory, a transceiver, and a battery or other suitable power source.

In aspects, in order to operate pen 20, the user first sets e.g., dials, a dose using a dose knob 26a of dose setting mechanism 25. For example, the dose may be adjusted up or down to achieve a desired dose amount prior to administration of the dose by rotating dose knob 26a in an appropriate direction. Once the appropriate dose has been set, the user applies a force against a dose dispensing button 26b of dose setting mechanism 25 to begin dispensing. More specifically, to begin dispensing, the user presses against the portion of dose dispensing button 26b that protrudes from body 22 of pen 20 to thereby drive a driving element 26c, e.g., a drive screw 26c, of dose dispensing mechanism 24 against an abutment of medicine cartridge 23 to dispense an amount of medicine from cartridge 23 through needle 29 into the user in accordance with the dose amount set by dose setting mechanism 25, e.g., dose knob 26a, during setting.

Operations monitoring mechanism 28 of pen 20 senses movement of a rotating and/or translating driving component (e.g., drive screw 26c) of dose dispensing mechanism 24. Operations monitoring mechanism 28 may include one or more switches, sensors, and/or encoders for this purpose. More specifically, any suitable switch(es), sensor(s), and/or encoder(s) may be utilized to sense rotary and/or linear movement. Non-limiting examples of such include rotary and linear encoders, Hall effect and other magnetic-based sensors, linearly variable displacement transducers, optical sensors, etc. With respect to an encoder, for example, the encoder can be configured to sense the rotation of drive screw 26c that, in turn, translates to dispense medicine; thus, by sensing rotation of drive screw 26c, the translational movement of drive screw 26c can be readily determined. Movement of the encoder may be detected as data processed by the processor of electronics unit 27 of pen 20, from which the amount of medicine dosed can be determined.

In aspects, the processor of electronics unit 27 of pen 20 can store the dose along with a timestamp for that dose and/or any other information associated with the dose. In aspects, the transceiver of electronics unit 27 enables pen 20 to transmit the dose and related information to computing device 30. In such aspects, once the dose is transmitted, the dose data and any related information associated with that particular transmitted dose is marked in the memory of electronics unit 27 of pen 20 as transmitted. If the dose is not yet transmitted to computing device 30 such as, for example, because no connection between the pen 20 and computing device 30 is available, then the dose and associated data can be saved and transmitted the next time a successful communication link between pen 20 and computing device 30 is established.

The timestamp may be the current time or a time from a count-up timer. When the dose and associated information is communicated to health management application 40 running on computing device 30, the timestamp and/or “time-since-dose” parameter (as determined by the count-up timer) is transmitted by pen 20 and received by computing device 30 for storage in memory 33 of data processing unit 31 of the computing device 30 (see FIG. 1C). Where a count-up timer is utilized, the time of the dose can be determined without pen 20 having to know the current time, which can simplify operation and setup of pen 20. That is, health management application 40 can determine the time of dose based on the current time and the value returned from the count-up timer.

Dose dispensing mechanism 24 of pen 20 can include a manually powered mechanism (user powered and/or mechanically biased), a motorized mechanism, or an assisted mechanism (e.g., a mechanism that operates partly on manual power and partly on motorized power). Regardless of the particular configuration of the dose dispensing mechanism 24, as noted above, when a force (e.g., a manual force, electrically-powered motor force, or combinations thereof) is applied to drive screw 26c of dose dispensing mechanism 24, drive screw 26c in turn provides a force to urge medicine from medicine cartridge 23 to deliver the set or dialed dose. In aspects, dose dispensing mechanism 24 can be operated such that rotation and/or translation of the driving element, e.g., drive screw 26c, is facilitated by a variable tension spring or a variable speed motor to inject the dose over a specific time frame (e.g., 1 s, 5 s, etc.) to help reduce the pain of dosing and/or for other purposes.

FIG. 1C illustrates computing device 30 (e.g., smartphone, tablet, wearable technology) of system 10 (FIG. 1A) including a data processing unit 31, a wireless communications unit 35, and a display unit 36. Data processing unit 31 includes a processor 32 to process data, a memory 33 in communication with the processor 32 to store data, and an input/output unit (I/O) 34 to interface processor 32 and/or memory 33 to other modules, units, and/or devices of computing device 30 and/or external devices. Processor 32 can include a central processing unit (CPU) or a microcontroller unit (MCU). Memory 33 can include and store processor-executable code, which when executed by processor 32, configures the data processing unit 31 to perform various operations, e.g., such as receiving information, commands, and/or data, processing information and data, and transmitting or providing information/data to another device. In aspects, data processing unit 31 can transmit raw or processed data to data processing system 50 (FIG. 1A). To support various functions of data processing unit 31, memory 33 can store information and data, such as instructions, software, values, images, and other data processed or referenced by processor 32. For example, various types of Random Access Memory (RAM) devices, Read Only Memory (ROM) devices, Flash Memory devices, and other suitable storage media can be used to implement storage functions of memory 33. I/O 34 of data processing unit 31 can interface data processing unit 31 with wireless communications unit 35 to utilize various types of wired or wireless interfaces compatible with typical data communication standards, for example, which can be used in communications of data processing unit 31 with other devices such as pen 20, via a wireless transmitter/receiver (Tx/Rx), e.g., including, but not limited to, Bluetooth, Bluetooth low energy, Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE/5G cellular communication methods, NFC (Near Field Communication), and parallel interfaces. I/O 34 of data processing unit 31 can also interface with other external interfaces, sources of data storage, and/or visual or audio display devices, etc. to retrieve and transfer data and information that can be processed by processor 32, stored in memory 33, and/or exhibited on an output unit of computing device 30 and/or an external device. For example, display unit 36 of computing device 30 can be configured to be in data communication with data processing unit 31, e.g., via I/O 34, to provide a visual display, an audio display, and/or other sensory display that produces the user interface of the health management application 40 (FIG. 1A). In some examples, display unit 36 can include various types of screen displays, speakers, or printing interfaces, e.g., including but not limited to, light emitting diode (LED), or liquid crystal display (LCD) monitor or screen, cathode ray tube (CRT) as a visual display; audio signal transducer apparatuses as an audio display; and/or toner, liquid inkjet, solid ink, dye sublimation, inkless (e.g., such as thermal or UV) printing apparatuses, etc.

Once computing device 30 receives the dose and related information (e.g., which can include time information, dose setting, and/or dose dispensing information, and other information about pen 20 and/or the environment and context as it relates to a dosing event), computing device 30 stores the dose related information in memory 33, e.g., which can be included among a list of doses or dosing events. In aspects, via the user interface associated with health management application 40, computing device 30 allows the user to browse a list of previous doses, to view an estimate of current medicine active in the patient's body (medicine on board, e.g., insulin on board) based on calculations performed by health management application 40, and/or to utilize a dose calculation module to assist the patient regarding dose setting information on the size of the next dose(s) to be delivered. For example, the patient may enter carbohydrates to be eaten and current blood sugar (which alternatively may be obtained directly from sensor device 60 (FIG. 1A)), and health management application 40 may already know insulin on board. Using these parameters, a suggested medicine dose (e.g., a recommended insulin dose), calculated by the dose determination module, may be determined. In aspects, computing device 30 can also allow the user to manually enter dose data, e.g., boluses, which may be useful if the battery in pen 20 has been depleted or another medicine delivery device, e.g., a syringe, was utilized to dose.

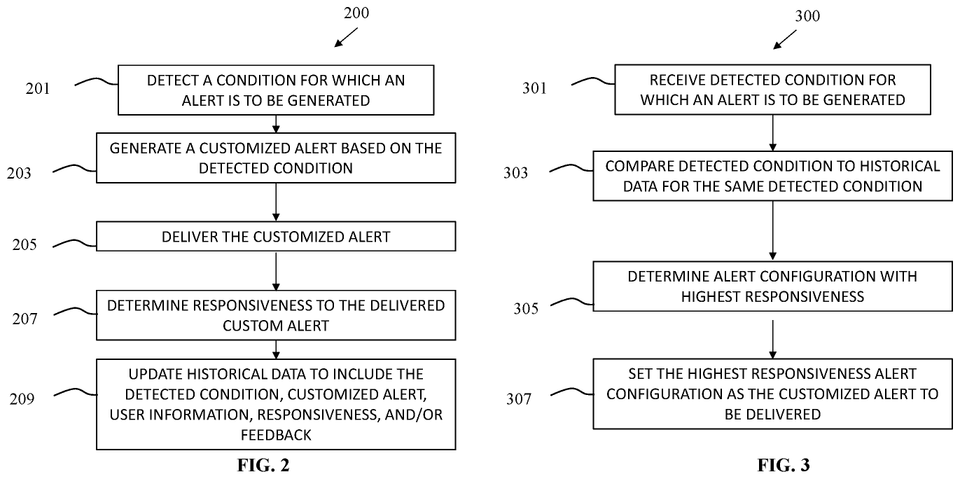

Turning now to FIG. 2, a method for customizing user alerts is illustrated as method 200. Method 200 (and any of the methods described herein) may be performed by any component or combination of components of system 10 (FIG. 1A); it is not intended for the steps of method 200 to be limited to being carried out by a single device or component. Additionally, while method 200 is described as including specific steps in a specific order, it is appreciated that method 200 may include some or all of the steps described, and the steps may be carried out in any order not specifically described. In an aspect, method 200 is performed (or stores instructions for being performed) by alert customization module 220 of health management application 40 (see FIG. 1C).

Method 200 begins at step 201 where a condition for which an alert is to be generated is detected. The alertable condition may be, for example, that the user should take a dose of medicine (e.g., an injection of insulin); that the user is due, overdue, etc. to take a scheduled dose of medicine; that the user should adjust a dose amount; that the user needs to confirm or input data; etc. The alertable condition may be detected based on a number of factors including, for example, a glucose concentration value (manually entered by the user or provided by a CGM) or Insulin-on-Board. The alertable condition may also be detected based on other data or input received or processed such as data received by computing device 30 including the tracked location of the user; physical activity of the user including step count, movement distance and/or intensity, estimated calories burned, and/or activity duration; and/or interaction pattern of the user with computing device 30. It is noted that the alertable condition need not be detected based on physiological factors but, rather, may additionally or alternatively be based on other factors such as time of day, time since last dose, etc.

After an alertable condition is detected in step 201, method 200 proceeds to step 203. In step 203, a customized alert for the alertable condition is generated for the user. The customized alert may be based at least in part on historical data of the user's responsiveness to past alerts. In particular, system 10 stores historical data corresponding to the user's historical responsiveness to different types of alerts, for different alertable conditions, delivered at different times and days, and/or under different circumstances. The customization of the alert may include any of the factors described below with respect to alert customization module 220 (FIG. 1C). For example, the customization of the alert may include but is not limited to, determining the type of device to alert, the manner in which the alert is delivered (e.g., audible, visual, haptic, and/or vibratory), the recipient(s) of the alert (e.g., patient-user, healthcare provider, parent(s) or guardian(s), school nurse, etc.), determining an output level for which the alert should be delivered (e.g., determining a volume level when the alert is audible, determining a color or brightness level when the alert is visual, determining an intensity or vibratory pattern when the alert is vibratory or haptic), and determining a time when the generated custom alert should be delivered (e.g., immediately, delayed a preconfigured period of time, etc.).

In step 205, the generated custom alert is delivered to the user (and/or other recipient(s) as determined in alert customization step 203). In step 207, a user's responsiveness to the generated custom alert is determined. A user's responsiveness to the generated custom alert may include, but is not limited to, a determination as to whether the generated custom alert was viewed and/or acknowledged, whether any action was taken in response to receipt of the generated custom alert, the result of the action taken, a comparison of the action taken to an action recommended in the alert, etc. If a user response is not received after a preconfigured period of time (or even in the event a response has been received or action taken), a prompt may be delivered to solicit a response of the user or to obtain feedback from the user pertaining to the reasons for the lack of acknowledgement of the delivered alert or other feedback regarding the alert, to be used for further alert customization for that user and/or other users. The preconfigured period of time may be customized for the particular user based on historical data of the user. In step 209, the historical data of the user is updated based on the determined response of the generated custom alert, the condition for which the result was triggered, user information, and/or the received feedback.

Referring to FIGS. 3-5, methods 300-500 for generating the customized alert, e.g., for step 203 of method 200 (FIG. 2), are described. It is noted that while methods 300-500 are detailed as distinct methods, they may be merged, performed sequentially, performed simultaneously, or in any other suitable manner. Each method 300, 400, 500 begins with receiving a detected condition for which an alert is to be generated at steps 301, 401, 501, respectively, e.g., as determined in step 201 of method 200 (FIG. 2). With particular reference to FIG. 3, in step 303 of method 300, the detected condition is compared to historical data for the same (or similar) detected condition. The returned historical data results are then reviewed to determine the alert configuration, among the returned results, that provided the highest and/or best responsiveness in step 307, and that customized alert is set, in step 309, as the customized alert to be delivered.

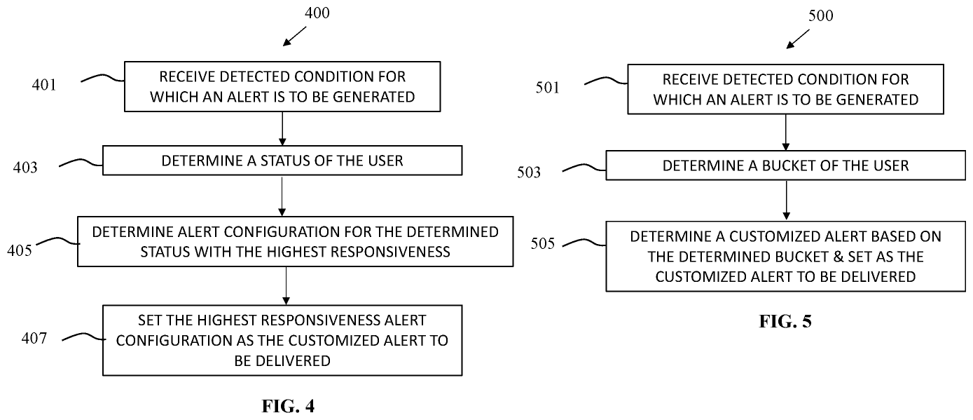

With particular reference to FIG. 4, after step 401 in method 400, the method proceeds to step 403, wherein a status of the user is determined. The status may be, for example, an occupation status of the user (e.g., whether the user is at work, school, home, on vacation, etc.), a social status of the user (e.g., whether the user is at home, at an event, driving, etc.), a medical status of the user (e.g., based on physiological data), and/or other statuses. The status may be determined based on input data, aggregated data from other connected devices, sensed data (such as movement data, geolocation data, gesture data, etc.), etc. In step 405, the alert configuration that provided the highest and/or best responsiveness, e.g., based on historical data of the user and/or other users, is determined. In step 407, the alert configuration determined to possess the highest and/or best responsiveness in step 405 is set as the customized alert to be delivered.

With particular reference to FIG. 5 after step 501 in method 500, the method proceeds to step 503, wherein a cluster of the user is determined. The clusters may be based on: users of similar demographics; users categorized based on attentiveness to alerts, compliance with their health plan, and/or other responsiveness factors; user preferences for alert configuration and/or frequency; user-selected settings; the user's selected cluster (or selection of a proxy for a particular cluster); and/or other considerations. Each cluster may include a pre-configured set of alert configurations, e.g., for each detected condition or group of detected conditions. Once the cluster is determined in step 503, the method proceeds to step 505, where the customized alert is determined based on the cluster and set as the customized alert to be delivered. For example, for the given detected condition and present cluster of the user, the corresponding alert configuration is set as the customized alert to be delivered.

Referring back to FIG. 1C, the alert customization module 220 of the health management application 40 is configured to perform some of all of the above-noted methods, facilitated, for example, and as mentioned above, by the receipt and processing of health data and user contextual data to calculate and customize a time-relevant and circumstance-relevant alert provided to the user. For example, the health data can include information pertaining to the medicine to dose (e.g., type of medicine in the pen 20 (FIG. 1A) to dose, such as type of insulin; previous amount or amounts dosed, such as last dose or a historical dose; and/or other medicines used by the user), information pertaining to a measured analyte (e.g., glucose value, including present glucose values, present glucose trend, and/or historical glucose values and trends), and other health-related information including heart rate, blood pressure, menstrual cycle, etc. For example, contextual data can include food intake (e.g., amount of carbs and/or total calories), physical activity (e.g., timing of activity or exercise, associated calories burned, etc.), the user's location (e.g., at a restaurant, gym, home, etc.), the user's mental state, and other data related to the user's behavior and lifestyle. The alert customization module 220 can receive data from one or more devices of the user, such as the pen 20 (FIG. 1A), an analyte sensor device 50 (FIG. 1A) such as a glucose meter (e.g., continuous glucose monitor (CGM) device or single measurement blood glucose meter (SMBG) device), and/or activity monitors (e.g., activity or exercise watch or smartwatch, pedometer, and the like). The alert customization module 220 can receive data from other software applications or modules resident on the computing device 30. The alert customization module 220 may additionally or alternatively dynamically factor user parameters that can be entered by a user (e.g., the user and/or an authorized user such as the user's physician or caregiver) and/or parameters determined from data tracked by the health management application 40 (FIG. 1A) (e.g., from the pen 20 (FIG. 1A) and/or pulled from another application such as a health aggregation application). In some implementations, the alert customization module 220 can determine a recommended dose of the medicine loaded in the pen device based on established clinical rules and/or aggregated data from larger populations (e.g., “crowdsourced” or clustered group data), using user-specific parameters such as weight, gender, ethnicity, and/or height (BMI), total daily dose, carb intake, physical activity, etc.

The alert customization module 220 is configured to compare metrics of exercise (e.g., steps), and if sufficient activity or exercise is detected, generate an output indicative of a warning to be provided to the user that their present dose setting (e.g., previous dose calculation) may suggest too many carbs due to their recent exercise. The alert customization module 220 is configured to use hypoglycemia as a metric or gain to the learning algorithm, e.g., to make user specific. For example, the alert customization module 220 can be configured to make variables more conservative until acceptable levels of hypoglycemia are reached.

In some implementations, the alert customization module 220 can produce an output indicative of recommending exercise, e.g., in lieu of insulin to lower a glucose level, based on the historical data for this user indicating that this user or the cluster of users is more receptive to exercise recommendations over administering a dose. For example, known exercise routines such as a 5 minute walk or 15 minutes of cardio may be calibrated for that particular user, based on past experience of how much such activities lower glucose for this particular user or other users within the clustered group. The example algorithm can base the calculated exercise recommendation on one or more of several factors, including insulin on board (JOB), food recently eaten, time of day, other recent exercise, and other parameters used by a calculation algorithm. The alert customization module 220 can track activity by monitoring accelerometer data, e.g., from the pen 20 itself, and/or from the computing device 30, and/or based on data from other wearable devices (such as activity monitors, for example) to adjust dose calculations/recommendations.

In some implementations, for example, the alert customization module 220 is configured to stagger partial-doses based on certain, user specific circumstances. In an illustrative example, the alert customization module 220 is configured stagger partial-doses based on foods with known slow glycemic response for this user of other users in the cluster. For example, pizza may prompt for a partial correction dose immediately and the rest of the dose an hour later to try to maintain a flat glucose response. This partial correction may be based on the user's own history with this or similar foods, and/or aggregated data from a larger population of users who have all had experience with this type of food. The alert customization module 220 can provide a dose reminder automatically set for (in this example) an hour later. In some implementations, for example, the alert customization module 220 is configured to also adjust a second dose based on glucose level, or prompt for a glucose measurement before recommending the second dose.

The various aspects and features disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described functional and/or operational aspects may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing unit” as used herein may refer to any of the foregoing structure or any other physical structure suitable for implementation of the described techniques. Also, the techniques could be fully implemented in one or more circuits or logic elements.

While several aspects of the present disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. A method for customizing user alerts in a medicine administration and tracking system, comprising:
detecting an alertable condition for which a medicine administration and tracking system is configured to monitor;
generating a custom alert for the alertable condition based at least in part on historical data indicating a responsiveness to previous alerts for the same alertable condition;
delivering the generated custom alert to a user;
determining the user's response to the generated custom alert; and
updating the historical data to include the alertable condition, the generated custom alert, and the user's level of responsiveness to the generated custom alert.

2. The method according to claim 1, wherein generating the custom alert includes determining at least one of a type of device to which the alert is to be delivered or a manner in which the alert is to be delivered.

3. The method according to claim 1, wherein the generated custom alert is at least one of:
an audible alert and generating the custom alert includes determining a volume level of the audible alert; or
a haptic alert and generating the custom alert includes determining an intensity or vibratory pattern of the haptic alert.

4. The method according to claim 1, wherein generating the custom alert includes delaying delivery of the alert to the user based on a time of day that the alertable condition is detected.

5. The method according to claim 1, further comprising delaying delivery of the custom alert based on an activity of the user.

6. The method according to claim 1, wherein the generated custom alert includes a calculated insulin dose recommendation.

7. The method according to claim 1, wherein generating the custom alert includes determining for what alert configuration the user is most likely to respond.

8. The method according to claim 1, wherein the user is clustered into a cluster and generating the custom alert is based on a setting associated with the cluster.

9. The method according to claim 1, wherein determining the user's response to the generated custom alert includes determining that the user has not responded to the generated custom alert after expiration of a predetermined period of time.

10. The method according to claim 9, further comprising:
prompting the user for feedback on the failure to respond; and
updating the historical data of the user based on the feedback.

11. A non-transitory computer-readable storage medium, storing instructions, which when executed by a processor, cause the processor to:
detect an alertable condition for which a medicine administration and tracking system is configured to monitor;
generate a custom alert for the alertable condition based at least in part on historical data indicating a responsiveness to previous alerts for the same alertable condition;
deliver the generated custom alert to a user;
determine the user's response to the generated custom alert; and
update the historical data to include the alertable condition, the generated custom alert, and the user's level of responsiveness to the generated custom alert.

12. The non-transitory computer-readable storage medium according to claim 11, wherein the instructions, when executed by the processor, cause the processor to generate the custom alert by determining at least one of a type of device to which the alert is to be delivered or a manner in which the alert is to be delivered.

13. The non-transitory computer-readable storage medium according to claim 11, wherein the generated custom alert is at least one of:
an audible alert and the instructions, when executed by the processor, cause the processor to determine a volume level of the audible alert; or
a haptic alert and the instructions, when executed by the processor, cause the processor to determine an intensity or vibratory pattern of the haptic alert.

14. The non-transitory computer-readable storage medium according to claim 11, wherein the instructions, when executed by the processor, cause the processor to generate the custom alert by delaying delivery of the alert to the user based on a time of day that the alertable condition is detected.

15. The non-transitory computer-readable storage medium according to claim 11, wherein the instructions, when executed by the processor, cause the processor to delay delivery of the alert based on an activity of the user.

16. The non-transitory computer-readable storage medium according to claim 11, wherein the generated custom alert includes a calculated insulin dose recommendation.

17. The non-transitory computer-readable storage medium according to claim 11, wherein the instructions, when executed by the processor, cause the processor to generate the custom alert by determining for what alert configuration the user is most likely to respond.

18. The non-transitory computer-readable storage medium according to claim 11, wherein the user is clustered into a cluster and the instructions, when executed by the processor, cause the processor to generate the custom alert based on a setting associated with the cluster.

19. The non-transitory computer-readable storage medium according to claim 11, wherein the instructions, when executed by the processor, cause the processor to determine the user's response to the alert by determining that the user has not responded to the generated custom alert after expiration of a predetermined period of time.

20. The non-transitory computer-readable storage medium according to claim 19, wherein the instructions, when executed by the processor, cause the processor to:
prompt the user for feedback on the failure to respond; and
update the historical data of the user based on the feedback.
